(19) United States
(12) Patent Application Publication
(10) Pub. No.: US 2025/0296899 A1
(43) Pub. Date: Sep. 25, 2025

(54) PROCESS TO UPGRADE AROMATIC WASTE STREAMS

(71) Applicant: Basell Poliolefine Italia S.r.l., Milan (IT)

(72) Inventors: David S. Mannel, Humble, TX (US); Sandor Nagy, Seabrook, TX (US); Barbara Kimmich, Houston, TX (US); Karl P. Rufener, Humble, TX (US); Daniel F. White, Houston, TX (US); Robert J. Casey, Kingwood, TX (US)

(73) Assignee: Basell Poliolefine Italia S.r.l., Milan (IT)

(21) Appl. No.: 19/086,434

(22) Filed: Mar. 21, 2025

Related U.S. Application Data
(60) Provisional application No. 63/568,581, filed on Mar. 22, 2024.

(51) Int. Cl. C07C 1/24; C07C 4/24; C07C 5/32; C07C 15/04

(52) U.S. Cl. CPC C07C 1/24; C07C 4/24; C07C 5/324; C07C 15/04

(57) ABSTRACT

Disclosed are methods for upgrading heavy aromatic waste streams, including polystyrene and other related materials, to chemical feedstocks. Process embodiments include feeding heavy aromatic waste streams to: a) hydrogenation followed by steam cracking followed by separation; and b) separation followed by: i) hydrogenation, and ii) hydrogenation followed by steam cracking followed by and separation. Each embodiment leads to production of ethylene, propylene, ethylbenzene, ethylbenzene precursors, or a combination thereof.

### CROSS-REFERENCE TO RELATED APPLICATIONS

This application claims priority of U.S. Provisional Patent Application Ser. No. 63/568,581, entitled “PROCESS TO UPGRADE AROMATIC WASTE STREAMS,” filed on Mar. 22, 2024, the contents of which are incorporated by reference herein in their entirety.

### FIELD OF THE INVENTION

This disclosure relates generally to the field of chemical recycling and more specifically to processes for recycling and converting heavy aromatic waste and/or fuel oil streams, including polystyrene and other related materials, back to chemical feedstocks.

### BACKGROUND OF THE INVENTION

The production of styrene and ethylbenzene is a critical aspect of the chemical industry, with applications spanning numerous products, including plastics, resins, and rubber. The Propylene Oxide/Styrene Monomer (POSM) process is a well-known technology for producing styrene and propylene oxide. However, the reliance on non-renewable resources and the generation of by-products and waste streams present environmental and economic challenges.

Polystyrene waste, a significant environmental concern due to its non-biodegradability, has traditionally been addressed through mechanical recycling processes. However, these processes are limited in their ability to achieve complete chemical circularity and often result in downcycling of the material. Furthermore, heavy aromatic waste streams, such as residual fuel oils (RFOs) and heavy aromatic solvents (HAS), are by-products of various chemical processes, including the POSM process itself. These waste streams are often underutilized and pose disposal and environmental issues.

There is a need for improved processes to recycle these waste streams back into valuable chemical feedstocks, thereby reducing reliance on virgin raw materials, enhancing the sustainability of chemical processes, and contributing to the circular economy. Ideally, such improved processes could be implemented in existing facilities by retrofitting or built into new facilities employing commonly used equipment and familiar techniques.

### SUMMARY OF THE INVENTION

In some embodiments, a process to upgrade a heavy aromatic waste stream to chemical feedstocks, comprises subjecting the heavy aromatic waste stream to hydrogenation conditions in a first reaction zone to produce a first intermediate product, wherein: i) the heavy aromatic waste stream comprises olefinic and aromatic unsaturation; and ii) the first intermediate product is substantially free of olefinic unsaturation and has a reduced amount of aromatic unsaturation. The process further comprises subjecting the first intermediate product to steam cracking conditions in a second reaction zone to produce a second intermediate product. The second intermediate product is then separated to produce a third intermediate product comprising ethylene, propylene, or a combination thereof and a first residual fraction.

In some embodiments, a process to upgrade a heavy aromatic waste stream to chemical feedstocks, comprises separating the heavy aromatic waste stream to produce a first intermediate product comprising styrene and a first residual fraction. The process further comprises subjecting the first intermediate product to hydrogenation conditions in a first reaction zone to produce a second intermediate product comprising ethylbenzene. The first residual fraction is then hydrogenated in a second reaction zone to produce a third intermediate product wherein: i) the first heavy fraction comprises olefinic and aromatic unsaturation; and ii) the third intermediate product is substantially free of olefinic unsaturation and has a reduced amount of aromatic unsaturation. The process further comprises subjecting the third intermediate product to steam cracking conditions in a third reaction zone to produce a fourth intermediate product. The fourth intermediate product is then separated to produce a fifth intermediate product comprising ethylene, propylene, or a combination thereof and a second residual fraction.

The foregoing has outlined rather broadly the features and technical advantages of the present invention in order that the detailed description of the invention that follows may be better understood. Additional features and advantages of the invention will be described hereinafter, which form the subject matter of the claims of the invention. It should be appreciated by those skilled in the art that the conception and specific embodiments disclosed may be readily utilized as a basis for modifying or designing other catalyst compositions and/or processes for carrying out the same purposes of the present invention. It should also be realized by those skilled in the art that such equivalent constructions do not depart from the spirit and scope of the invention as set forth in the appended claims. The novel features which are believed to be characteristic of the invention, both as to its compositions and processes, together with further objects and advantages will be better understood from the following description.

### BRIEF DESCRIPTION OF THE FIGURES

The claimed subject matter may be understood by reference to the following description taken in conjunction with the accompanying drawings, in which like reference numerals identify like elements, and in which:

FIG. 1 is a simplified flow diagram illustrating hydrogenation of heavy aromatic waste feed to remove olefinic and aromatic unsaturation to produce a first intermediate product, steam cracking of the first intermediate product to produce a second intermediate product comprising ethylene, and/or propylene, and separation of the ethylene and/or propylene suitable as feedstock or feedstock precursor for the POSM process and/or SM production process, according to embodiments of the disclosure; and

FIG. 2 is a simplified flow diagram illustrating separation of heavy aromatic waste feed to produce a first intermediate product having an increased styrene content and a first residual product, hydrogenation of the first intermediate product to produce EB suitable as feedstock to propylene oxide/styrene monomer (POSM) process or for direct conversion to styrene monomer (SM) via dehydrogenation, hydrogenation of the first residual product to remove olefinic and aromatic unsaturation to produce a second intermediate product, steam cracking of the second intermediate product to produce a third intermediate product comprising ethylene, and/or propylene, and separation of the ethylene and/or propylene suitable as feedstock or feedstock precursor for the POSM process and/or SM production process, according to embodiments of the disclosure.

While the disclosed process and composition are susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the subject matter claimed below will now be disclosed. In the interest of clarity, some features of some actual implementations may not be described in this specification. It will be appreciated that in the development of any such actual embodiments, numerous implementation-specific decisions must be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

### Definitions

As used herein, “a” or “an” when used in conjunction with the term “comprising” in the claims or the specification means one or more than one, unless the context dictates otherwise.

As used herein, “about” means the stated value plus or minus the margin of error of measurement or plus or minus 10% if no process of measurement is indicated.

As used herein, “consisting essentially of” excludes additional material elements, but allows the inclusions of non-material elements that do not substantially change the nature of the invention.

As used herein, “consisting of” is closed and excludes all additional elements.

As used herein, “conversion” is used to denote the percentage of a component fed which disappears across a reactor.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used herein, “reaction zone” refers to a chamber sufficiently enclosed to maintain selected operating conditions within the chamber to produce a desired reaction, such as a pyrolysis reaction zone, a steam cracking reaction zone, a catalytic cracking reaction zone, or a hydrogenation reaction zone. In some embodiments, each reaction zone can be a separate reactor. In some embodiments, a single vessel can contain a plurality of reaction zones.

As used herein, “separation conditions” or “separation section” means facilities including distillation, absorption, adsorption, membrane separation, cryogenic distillation, extractive distillation, azeotropic distillation, steam distillation, molecular sieves, liquid-liquid extraction (solvent extraction), decantation, centrifugation, or a combination thereof as required to recover specific materials from intermediate products and/or reaction products disclosed herein. Such embodiments would include common equipment associated with the forgoing separation processes, including, but not limited to, columns, drums, vessels, heat exchangers, pumps, valves, reflux loops, and the like, the descriptions of which are omitted herein for simplicity. Where intermediate products and/or reaction products disclosed herein are defined as comprising multiple products (e.g., benzene, ethylene, propylene, or a combination thereof), it is intended that “separation conditions” or “separation section” includes the functionality to recover each of those products separately at a desired purity (e.g., 99 wt % benzene, 99.5 wt % ethylene, 97 wt % propylene, etc.) as required by the disposition of the product as a feed to various locations of other processes, such as, but not limited to, POSM and/or SM.

As used herein, “waste stream” is a type of feed stream comprising material that has been discarded as no longer useful, including but not limited to, post-consumer and post-industrial waste.

As used herein, “zeolite” refers to an aluminosilicate mineral with a microporous structure. Zeolites are, in one aspect, useful as catalysts for the processes disclosed herein. Zeolites can occur naturally or can be produced industrially.

All concentrations herein are by weight percent (“wt %”) unless otherwise specified.

The following abbreviations are used herein:

| Abbreviation | Term |
|---|---|
| ECR | ethylene cracking residue |
| HAS | heavy aromatic solvent |
| PAH | polycyclic aromatic hydrocarbons |
| PFO | pyrolysis fuel oil |
| POSM | Propylene oxide/styrene monomer production process |
| RFO | residual fuel oils |
| SM | Styrene monomer or process to produce styrene monomer, |
| | depending on the context |
| WHSV | weight hourly space velocity |

### Heavy Aromatic Waste

The present disclosure provides a process for upgrading aromatic waste, including polystyrene, into light olefins (e.g., ethylene and/or propylene), ethylbenzene, and/or ethylbenzene precursors useful as feedstocks to petrochemical processes, including, but not limited to, the propylene oxide/styrene monomer process and dehydrogenation, oxidative or non-oxidative, of ethylbenzene to produce styrene. Heavy aromatic waste streams include, but are not limited to, polystyrene pyrolysis oil, styrene, polystyrene pyrolysis oil heavies, heavy residual fuel oils (RFO), heavy aromatic solvent (HAS), pyrolysis fuel oil (PFO), and ethylene cracking residue (ECR). Heavy aromatic waste streams to be treated by the processes disclosed herein can be any one of the foregoing materials or any mixture of two or more of the foregoing materials.

Polystyrene pyrolysis involves the thermal degradation of polystyrene, a common thermoplastic material, in the absence of oxygen. This process breaks down the long polymer chains of polystyrene back into shorter hydrocarbon chains and styrene monomer, along with a range of other hydrocarbons. The products of polystyrene pyrolysis can be broadly categorized into two groups: polystyrene pyrolysis oil and polystyrene pyrolysis oil heavies. Each of these categories has distinct characteristics and potential applications. Polystyrene pyrolysis oil primarily consists of styrene monomer, which can be recovered and purified for reuse in the production of new polystyrene products or other styrenic polymers. Besides styrene, the oil contains a mixture of other aromatic hydrocarbons such as toluene, ethylbenzene, and benzene, as well as aliphatic hydrocarbons. The exact composition of the pyrolysis oil can vary based on the pyrolysis conditions such as temperature, heating rate, and the presence of catalysts. Polystyrene pyrolysis oil heavies consist of the heavier, more complex hydrocarbons that are produced during the pyrolysis process. These compounds are higher in molecular weight compared to the main fraction of pyrolysis oil and often include polycyclic aromatic hydrocarbons (PAH), along with various oligomers formed by partial recombination of degradation products. Pyrolysis of polystyrene presents an opportunity for recycling a plastic that is otherwise difficult to process through mechanical recycling methods. By converting waste polystyrene into valuable chemicals and fuels, pyrolysis can reduce landfilling and incineration, contributing to circular economy initiatives.

During the POSM process, various by-products are generated, including residual fuel oils (RFO) rich in aromatics. These RFOs are complex mixtures containing high molecular weight hydrocarbons, predominantly aromatics, along with aliphatics and small amounts of olefins. The aromatic content gives these oils their distinct characteristics, including high density and a high calorific value. The exact composition of these oils can vary depending on the specifics of the POSM process and the feedstocks used. These oils have high boiling points due to the presence of large, complex hydrocarbon molecules. The high aromatic content contributes to a higher density and viscosity compared to lighter fuel oils. While these oils are rich in energy content, their high aromatic content may affect their combustion characteristics. Depending on the feedstock and process conditions, the sulfur content can vary, potentially requiring desulfurization treatments for certain uses. Such RFOs have utility as components in fuel oil and asphalt blending but incur some safety, environmental, and regulatory considerations due to high aromatics content and/or emissions such as NOx, SOx, and particulate matter.

Benzene is typically converted to ethylbenzene by reacting benzene with ethylene in an alkylation process. Heavy aromatic solvents (HAS) can be produced as by-products of the alkylation process. These heavy aromatic solvents are generally composed of higher molecular weight aromatic compounds that form through side reactions during the alkylation process. A common HAS produced in this context is diethylbenzene (DEB), which consists of three isomers: ortho-, meta-, and para-diethylbenzene. Diethylbenzene forms when an ethyl group is added to ethylbenzene in a subsequent alkylation reaction, essentially representing an over-alkylation of benzene. Although DEB has some direct uses, it would be desirable to break down and convert these heavy molecules into basic monomers suitable as feedstocks to a variety of petrochemical processes, including, but not limited to, POSM and EB dehydrogenation to SM.

In steam cracking processes, complex mixtures of by-products are generated in addition to producing olefins (e.g., ethylene, propylene). Such by-products include heavy aromatic-rich higher molecular weight hydrocarbons such as pyrolysis fuel oil (PFO) and ethylene cracking residue (ECR). PFO is a complex mixture that includes heavy aromatics, asphaltenes, and other hydrocarbons produced during the thermal cracking of naphtha or gas oils. Its exact composition varies depending on the feedstock and the operating conditions of the cracker but is characterized by its high content of polycyclic aromatic hydrocarbons (PAHs). ECR is the residue left from the steam cracking process, particularly when heavier feedstocks are used. It is rich in heavy aromatics, tar, and coke precursors. Like PFO, its specific composition depends on the feedstock and process conditions. Similarly to RFOs, PFO and ECR have utility as components in fuel oil but incur some safety, environmental, and regulatory considerations due to high aromatics content and/or emissions such as NOx, SOx, and particulate matter.

The present disclosure provides embodiments of processes to upgrade heavy aromatic waste streams, including polystyrene and other related materials, to chemical feedstocks. In some embodiments, the heavy aromatic waste stream comprises polystyrene pyrolysis oil, styrene, polystyrene pyrolysis oil heavies, heavy residual fuel oils (RFO), heavy aromatic solvent (HAS), pyrolysis fuel oil (PFO), ethylene cracking residue (ECR), or a combination thereof. In some embodiments, the heavy aromatic waste stream comprises styrene in an amount greater than or equal to 30 wt %, greater than or equal to 40 wt %, greater than or equal to 50 wt %, or greater than or equal to 60 wt %, based on the total weight of the heavy aromatic waste stream.

### Process Comprising Steam Cracking/Hydrogenation

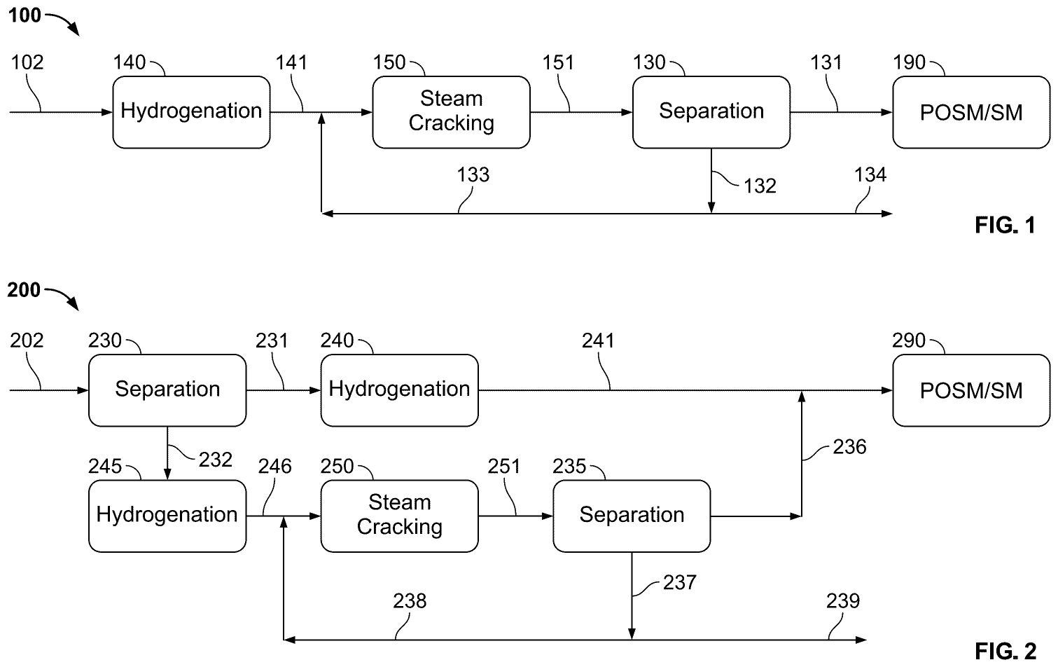

FIG. 1 shows a process 100 to upgrade a heavy aromatic waste stream 102 into chemical feedstocks. The heavy aromatic waste stream 102 has a first styrene content. The process conditions and catalyst selection are optimized to achieve a desired product composition. In some embodiments, the process 100 comprises subjecting the heavy aromatic waste stream 102 to hydrogenation conditions in a first reaction zone 140 to produce a first intermediate product 141, wherein the heavy aromatic waste stream 102 comprises olefinic and aromatic unsaturation, and the first intermediate product 141 is substantially free of olefinic unsaturation and has a reduced amount of aromatic unsaturation. Examples of hydrogenation catalysts include any commercial hydrogenation catalysts known in the art such as NiMo, CoMo, Ni, Pd, Pt, Cu, and the like. The first intermediate product 141 is subjected to steam cracking conditions in a second reaction zone 150 to produce a second intermediate product 151. Steam cracking is described in U.S. Pat. Nos. 2,847,366, 3,597,494, 4,832,822, 4,780,196, and 11,046,898; U.S. Publ. App. Nos. 2011/0073524, 2014/0083906, and 2008/0194900, the disclosures of which is fully incorporated by reference herein. The second intermediate product 151 is subjected to separation conditions 130 to produce a third intermediate product 131 comprising ethylene, propylene, or a combination thereof and a first residual fraction 132.

The third intermediate product 131 is then fed to a POSM/SM process 190, wherein POSM/SM means the POSM process and/or the SM process. SM process means the direct dehydrogenation of EB to form styrene monomer. The third intermediate product 131 comprises one or more of styrene, benzene, ethylene, and propylene. As part of POSM/SM process 190, one or more of styrene, benzene, ethylene, and propylene is recovered from the third intermediate product 131, wherein the purity of the recovered styrene, benzene, ethylene, and propylene is suitable for direct or indirect addition to the POSM and/or the SM process. Indirect addition means that one or more additional processing steps can be performed on the recovered stream to make it suitable for addition to the POSM and/or the SM process. The specific components in the heavy aromatic waste stream 102, the specific hydrogenation conditions in the first reaction zone 140, the specific steam cracking conditions in the second reaction zone 150, and the specific separation conditions in the separation section 130 will result in different amounts of styrene, benzene, ethylene, and/or propylene in the third intermediate product 131. Examples of hydrogenation catalysts include any commercial hydrogenation catalysts known in the art such as NiMo, CoMo, Ni, Pd, Pt, Cu, and the like.

In some embodiments, styrene is recovered from the third intermediate product 131. The styrene is hydrogenated to form EB. Examples of hydrogenation catalysts include any commercial hydrogenation catalysts known in the art such as NiMo, CoMo, Ni, Pd, Pt, Cu, and the like. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, benzene is recovered from the third intermediate product 131. The benzene is reacted with ethylene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, ethylene is recovered from the third intermediate product 131. The ethylene is reacted with benzene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, propylene is recovered from the third intermediate product 131. The propylene is added to the POSM process, wherein it is reacted with ethylbenzene hydroperoxide to form propylene oxide and 1-phenyl ethanol. The 1-phenyl ethanol is then dehydrated to produce styrene monomer.

In some embodiments, the first residual fraction 132 is added to the second reaction zone 150 as additional feed via stream 133, sent outside the process for addition processing via stream 134, or a combination thereof.

FIG. 2 shows a process 200 to upgrade a heavy aromatic waste stream 202 into chemical feedstocks. The heavy aromatic waste stream 202 has a first styrene content, a first benzene content, a first ethylene content, a first propylene content, or a combination thereof. The process 200 comprises subjecting the heavy aromatic waste stream 202 to separation conditions 230 to produce a first intermediate product 231 and a first residual fraction 232. The process conditions and catalyst selection are optimized to achieve a desired product composition. In some embodiments, the first intermediate product 231 has a second styrene content less than the first styrene content, a second benzene content less than the first benzene content, a second ethylene content less than the first styrene content, a second propylene content less than the first propylene content, or a combination thereof. The first intermediate product 231 is subjected to hydrogenation conditions in a first reaction zone 240 to produce a second intermediate product 241 comprising EB. Examples of hydrogenation catalysts include any commercial hydrogenation catalysts known in the art such as NiMo, CoMo, Ni, Pd, Pt, Cu, and the like. EB is recovered from the second intermediate product 241. At least a portion of the EB is: a) oxidized to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer; b) dehydrogenated to produce styrene monomer; or c) a combination thereof.

The first residual fraction 232 is subjected to hydrogenation conditions in a second reaction zone 245 to produce a third intermediate product 246, wherein the first residual fraction 232 comprises olefinic and aromatic unsaturation, and the third intermediate product 246 is substantially free of olefinic unsaturation and has a reduced amount of aromatic unsaturation. Examples of hydrogenation catalysts include any commercial hydrogenation catalysts known in the art such as NiMo, CoMo, Ni, Pd, Pt, Cu, and the like. The third intermediate product 246 is subjected to steam cracking conditions in a third reaction zone 250 to produce a fourth intermediate product 251. The fourth intermediate product 251 is subjected to separation conditions 235 to produce a fifth intermediate product 236 comprising ethylene, propylene, benzene, or a combination thereof and a second residual fraction 237.

In some embodiments, benzene is recovered from the fifth intermediate product 236. The benzene is reacted with ethylene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, ethylene is recovered from the fifth intermediate product 236. The ethylene is reacted with benzene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, propylene is recovered from the fifth intermediate product 236. The propylene is added to the POSM process, wherein it is reacted with ethylbenzene hydroperoxide to form propylene oxide and 1-phenyl ethanol. The 1-phenyl ethanol is then dehydrated to produce styrene monomer.

The third intermediate product 231 is then fed to a POSM/SM process 290, wherein POSM/SM means the POSM process and/or the SM process. SM process means the direct dehydrogenation of EB to form styrene monomer. The third intermediate product 231 comprises one or more of styrene, benzene, ethylene, and propylene. As part of POSM/SM process 290, one or more of styrene, benzene, ethylene, and propylene is recovered from the third intermediate product 231, wherein the purity of the recovered styrene, benzene, ethylene, and propylene is suitable for direct or indirect addition to the POSM and/or the SM process. Indirect addition means that one or more additional processing steps can be performed on the recovered stream to make it suitable for addition to the POSM and/or the SM process. The specific components in the heavy aromatic waste stream 202, the specific hydrogenation conditions in the first reaction zone 240, the specific steam cracking conditions in the second reaction zone 250, and the specific separation conditions in the separation section 230 will result in different amounts of styrene, benzene, ethylene, and/or propylene in the third intermediate product 231. Examples of hydrogenation catalysts include any commercial hydrogenation catalysts known in the art such as NiMo, CoMo, Ni, Pd, Pt, Cu, and the like.

In some embodiments, styrene is recovered from the third intermediate product 231. The styrene is hydrogenated to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, benzene is recovered from the third intermediate product 231. The benzene is reacted with ethylene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, ethylene is recovered from the third intermediate product 231. The ethylene is reacted with benzene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, propylene is recovered from the third intermediate product 231. The propylene is added to the POSM process, wherein it is reacted with ethylbenzene hydroperoxide to form propylene oxide and 1-phenyl ethanol. The 1-phenyl ethanol is then dehydrated to produce styrene monomer.

In some embodiments, the second residual fraction 237 is added to the third reaction zone 250 as additional feed via stream 238, sent outside the process for addition processing via stream 239, or a combination thereof.

### Certain Embodiments

Embodiment A1. A process to upgrade a heavy aromatic waste stream to chemical feedstocks, the process comprising:
a) subjecting the heavy aromatic waste stream to hydrogenation conditions in a first reaction zone to produce a first intermediate product, wherein:
   i) the heavy aromatic waste stream comprises olefinic and aromatic unsaturation; and
   ii) the first intermediate product is substantially free of olefinic unsaturation and has a reduced amount of aromatic unsaturation;
b) subjecting the first intermediate product to steam cracking conditions in a second reaction zone to produce a second intermediate product; and
c) separating the second intermediate product to produce a third intermediate product comprising ethylene, propylene, or a combination thereof and a first residual fraction. Embodiment A2. The process of Embodiment A1, further comprising:

a) recovering the ethylene from the third intermediate product; and

b) reacting the ethylene with benzene to form ethylbenzene.

Embodiment A3. The process of Embodiment A1 or A2, further comprising:
a) oxidizing at least a portion of the ethylbenzene to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene to produce styrene monomer; or
c) a combination thereof.

Embodiment A4. The process of any one of Embodiments A1-A3, further comprising:
a) recovering the propylene from the third intermediate product;
b) catalytically reacting the propylene with ethylbenzene hydroperoxide to form propylene oxide and 1-phenyl ethanol; and
c) dehydrating the 1-phenyl ethanol to produce styrene monomer.

Embodiment A5. The process of any one of Embodiments A1-A4, further comprising:
a) adding at least a portion of the first residual fraction to the second reaction zone as additional feed;
b) sending at least a portion of the first residual fraction outside the process for further processing; or
c) a combination thereof.

Embodiment B1. A process to upgrade a heavy aromatic waste stream to chemical feedstocks, the process comprising:
a) separating the heavy aromatic waste stream to produce a first intermediate product comprising styrene and a first residual fraction;
b) subjecting the first intermediate product to hydrogenation conditions in a first reaction zone to produce a second intermediate product comprising ethylbenzene;
c) subjecting the first residual fraction to hydrogenation conditions in a second reaction zone to produce a third intermediate product wherein:
   i) the first heavy fraction comprises olefinic and aromatic unsaturation; and
   ii) the third intermediate product is substantially free of olefinic unsaturation and has a reduced amount of aromatic unsaturation;
d) subjecting the third intermediate product to steam cracking conditions in a third reaction zone to produce a fourth intermediate product; and
e) separating the fourth intermediate product to produce a fifth intermediate product comprising benzene, ethylene, propylene, or a combination thereof and a second residual fraction.

Embodiment B2. The process of Embodiment B1, further comprising recovering the ethylbenzene from the second intermediate product and:
a) oxidizing at least a portion of the ethylbenzene to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene to produce styrene monomer; or
c) a combination thereof.

Embodiment B3. The process of Embodiment B1 or B2, further comprising:
a) recovering the ethylene from the fifth intermediate product; and
b) reacting the ethylene with benzene to form ethylbenzene.

Embodiment B4. The process of any one of Embodiments B1-B3, further comprising:
a) oxidizing the ethylbenzene to form ethylbenzene hydroperoxide;
b) catalytically reacting the ethylbenzene hydroperoxide with propylene to form propylene oxide and 1-phenyl ethanol; and
c) dehydrating the 1-phenyl ethanol to produce styrene monomer.

Embodiment B5. The process of any one of Embodiments B1-B4, further comprising:
a) recovering the propylene from the fifth intermediate product;
b) catalytically reacting the propylene with ethylbenzene hydroperoxide to form propylene oxide and 1-phenyl ethanol; and
c) dehydrating the 1-phenyl ethanol to produce styrene monomer.

Embodiment B6. The process of any one of Embodiments B1-B5, further comprising:
a) recovering the benzene from the third intermediate product or the fourth intermediate product; and
b) reacting the benzene with ethylene to form ethylbenzene.

Embodiment B7. The process of Embodiment B6, further comprising:
a) oxidizing at least a portion of the ethylbenzene to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene to produce styrene monomer; or
c) a combination thereof.

Embodiment B8. The process of any one of Embodiments B1-B7, further comprising:
a) adding at least a portion of the second residual fraction to the third reaction zone as additional feed;
b) sending at least a portion of the first residual fraction outside the process for further processing; or
c) a combination thereof.

The presently disclosed processes are exemplified in the examples below. These examples are included to demonstrate embodiments of the appended claims. However, these are exemplary only, and the invention can be broadly applied to embodiments not demonstrated in the examples. Those of skill in the art should appreciate that many changes can be made in the specific embodiments which are disclosed and still obtain a like or similar result without departing from the spirit and scope of the disclosure herein. In no way should the following examples be read to limit, or to define, the scope of the appended claims.

### EXAMPLES

The following examples are included to demonstrate embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Background

Characterization of liquid products: The liquid products from the two traps were characterized by Gas Chromatography (GC) and proton NMR (¹H NMR).

The GC analysis of the liquid product for each run was performed using an Agilent 7890 GC (Agilent Technologies, Santa Clara, CA) equipped with a standard non-polar column and a flame ionization detector. For the GC data, the weight percent for x<nC7 (having boiling point <98° C. named LF1), nC7<x<nC11 (having boiling point 98° C.<BP<203° C. named LF2), nC12<x<nC28 (having boiling point 203° C.<BP<434° C. named LF3), x>C28 (having boiling point >434° C. named LF4) were used to characterize the liquid product.

NMR data were used to characterize the percent of aromatic protons, paraffinic protons and olefinic protons in the liquid product. The examples were analyzed with an addition of CDCl3 (0.6 g of depolymerize polymer/metal oxide mixture with 0.4 g of CDCl3). The data were collected on a Bruker AV500 MHz NMR spectrometer (Bruker Corporation, Billerica, MA) at 25° C. with a 5 mm Prodigy probe. One dimension 1H NMR data were processed using TOPSPIN® software (Bruker) with an exponential line broadening window function. Quantitative measurements were performed with a 15 second relaxation delay, a 30° flip angle pulse, and 32 scans to facilitate accurate integrals. The spectral integrations for aromatic olefinic, and paraffinic protons were obtained and used to quantify relative ratios of these protons.

### FTIR Process

FTIR spectroscopy was carried out as follows. An iS50 FTIR spectrometer equipped with a DTGS detector from Thermo Scientific was used for FTIR spectral acquisition. The sample compartment was fitted with an attenuated total reflectance (ATR) accessory. The ATR cell was obtained from Pike Technologies and was equipped with a 3-bounce zinc selenide (ZnSe) crystal. A sample volume of about 0.05 mLs was required for analysis. Those familiar with FTIR spectroscopy will recognize that when multiple spectra are overlaid in the same plot, these spectra can be shown with similar baselines or offset baselines. This choice is purely for optimal viewing of changes occurring and does not impact peak height or peak absorbance measurements as such measurements are made from the baseline to the top of the peak.

### Hydrogenation

Table 3 shows effectiveness of hydrogenating pyrolysis product of polystyrene pyrolysis heavies. ¹H NMR measurements of a sample of pyrolysis product of polystyrene are shown before and after hydrogenation using NiMo-S (KL-8234) catalyst. Results show a reduction in olefinic and aromatic double bonds after hydrogenation.

TABLE 3

| | | | | Styrene | | |
|---|---|---|---|---|---|---|
| | | | | double | | |
| Sample | Saturated | Oxygenates | Olefinic | bond | Aromatic | Aldehyde |
| Starting | 62.002 | 2.159 | 2.7475 | 1.5939 | 31.3861 | 0.1114 |
| Material | | | | | | |
| Hydrogenated | 71.315 | 0.9257 | 0.463 | 0.1905 | 27.1028 | 0.003 |
| Material | | | | | | |

Table 4 shows the yield of the product composition resulting from hydrogenation of a liquid feed comprising 10 wt % pyrolysis oil made from the thermal pyrolysis of polystyrene and 90 wt % decalin used as a diluent. A stainless steel continuous packed bed reactor loaded with 3 grams of a sulfided NiMo catalyst (NiMo-S) was used to hydrogenate the liquid feed flowing through the catalyst bed at a flow rate of 0.025 mL/min with a hydrogen (H2) gas flow rate of 100 sccm. Results show complete conversion of styrene with greater than 98% of the styrene present in the feed being converted to ethylbenzene. The GC analysis of the product composition was performed using an Agilent 7890 GC (Agilent Technologies, Santa Clara, CA) equipped with a standard non-polar column and a flame ionization detector. For the GC data, the weight percent for x<nC7 (having boiling point <98° C. named LF1), nC7<x<nC11 (having boiling point 98° C. <BP<203° C. named LF2), nC12<x<nC28 (having boiling point 203° C.<BP<434° C. named LF3), x>C28 (having boiling point >434° C. named LF4) were used to characterize the product.

TABLE 4

| Component | Ethylbenzene | Ethylcyclohexane | Styrene | Heavies |
|---|---|---|---|---|
| Yield (mol %) | 60% | 1% | 0% | 39% |

For the sake of brevity, only certain ranges are explicitly disclosed herein. However, in addition to recited ranges, any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the processes, machines, means, processes, and/or steps described in the specification. As one of the ordinary skill in the art will readily appreciate from the disclosure of the present invention, processes, machines, means, processes, and/or steps, presently existing or later to be developed that perform substantially the same function or achieve substantially the same result as the corresponding embodiments described herein, may be utilized according to the present invention. Accordingly, the appended claims are intended to include within their scope such processes, machines, means, processes, and/or steps.

What is claimed is:

1. A process to upgrade a heavy aromatic waste stream to chemical feedstocks, the process comprising:
a) subjecting the heavy aromatic waste stream to hydrogenation conditions in a first reaction zone to produce a first intermediate product, wherein:
i) the heavy aromatic waste stream comprises olefinic and aromatic unsaturation; and
ii) the first intermediate product is substantially free of olefinic unsaturation and has a reduced amount of aromatic unsaturation;
c) subjecting the first intermediate product to steam cracking conditions in a second reaction zone to produce a second intermediate product; and
d) separating the second intermediate product to produce a third intermediate product comprising ethylene, propylene, or a combination thereof and a first residual fraction.

2. The process of claim 1, further comprising:
a) recovering the ethylene from the third intermediate product; and
b) reacting the ethylene with benzene to form ethylbenzene.

3. The process of claim 2, further comprising:
a) oxidizing at least a portion of the ethylbenzene to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene to produce styrene monomer; or
c) a combination thereof.

4. The process of claim 1, further comprising:
a) recovering the propylene from the third intermediate product;
b) catalytically reacting the propylene with ethylbenzene hydroperoxide to form propylene oxide and 1-phenyl ethanol; and
c) dehydrating the 1-phenyl ethanol to produce styrene monomer.

5. The process of claim 1, further comprising:
a) adding at least a portion of the first residual fraction to the second reaction zone as additional feed;
b) sending at least a portion of the first residual fraction outside the process for further processing; or
c) a combination thereof.

6. A process to upgrade a heavy aromatic waste stream to chemical feedstocks, the process comprising:
a) separating the heavy aromatic waste stream to produce a first intermediate product comprising styrene and a first residual fraction;
b) subjecting the first intermediate product to hydrogenation conditions in a first reaction zone to produce a second intermediate product comprising ethylbenzene;
c) subjecting the first residual fraction to hydrogenation conditions in a second reaction zone to produce a third intermediate product wherein:
i) the first heavy fraction comprises olefinic and aromatic unsaturation; and
ii) the third intermediate product is substantially free of olefinic unsaturation and has a reduced amount of aromatic unsaturation;
d) subjecting the third intermediate product to steam cracking conditions in a third reaction zone to produce a fourth intermediate product; and
e) separating the fourth intermediate product to produce a fifth intermediate product comprising benzene, ethylene, propylene, or a combination thereof and a second residual fraction.

7. The process of claim 6, further comprising recovering the ethylbenzene from the second intermediate product and:
a) oxidizing at least a portion of the ethylbenzene to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene to produce styrene monomer; or
c) a combination thereof.

8. The process of claim 6, further comprising:
a) recovering the ethylene from the fifth intermediate product; and
b) reacting the ethylene with benzene to form ethylbenzene.

9. The process of claim 8, further comprising:
a) oxidizing the ethylbenzene to form ethylbenzene hydroperoxide;
b) catalytically reacting the ethylbenzene hydroperoxide with propylene to form propylene oxide and 1-phenyl ethanol; and
c) dehydrating the 1-phenyl ethanol to produce styrene monomer.

10. The process of claim 6, further comprising:
a) recovering the propylene from the fifth intermediate product;
b) catalytically reacting the propylene with ethylbenzene hydroperoxide to form propylene oxide and 1-phenyl ethanol; and
c) dehydrating the 1-phenyl ethanol to produce styrene monomer.

11. The process of claim 6, further comprising:
a) recovering the benzene from the third intermediate product or the fourth intermediate product; and
b) reacting the benzene with ethylene to form ethylbenzene.

12. The process of claim 11, further comprising:
a) oxidizing at least a portion of the ethylbenzene to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene to produce styrene monomer, or
c) a combination thereof.

13. The process of claim 6, further comprising:
a) adding at least a portion of the second residual fraction to the third reaction zone as additional feed;
b) sending at least a portion of the first residual fraction outside the process for further processing; or
c) a combination thereof.
